(19) United States
(12) Patent Application Publication
(10) Pub. No.: US 2007/0287716 A1
(43) Pub. Date: Dec. 13, 2007

(54) Pyrimidine and Quinoline Potentiators of Metabotropic Glutamate Receptors

(76) Inventors: Essa H. Hu, San Diego, CA (US); Jean-Michel Vernier, San Diego, CA (US); Peter Chua, San Diego, CA (US); John H. Hutchinson, La Jolla, CA (US); Nicholas Cosford, San Diego, CA (US)

Correspondence Address: MERCK AND CO., INC, P O BOX 2000, RAHWAY, NJ, 07065-0907, US

(21) Appl. No.: 11/665,422

(22) PCT Filed: Oct. 24, 2005
(86) PCT No.: PCT/US05/38435

§ 371 Date: Apr. 12, 2007

Related U.S. Application Data
(60) Provisional application No. 60/622,868, filed on Oct. 28, 2004.

(51) Int. Cl. A61K 31/47; A61P 25/00; C07D 215/12; C07D 239/28

(52) U.S. Cl. 514/256; 514/311; 544/335; 546/176

(57) ABSTRACT

The present invention is directed to compounds which are potentiators of metabotropic glutamate receptors, including the mGluR2 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### BACKGROUND OF THE INVENTION

The excitatory amino acid L-glutamate (sometimes referred to herein simply as glutamate) through its many receptors mediates most of the excitatory neurotransmission within the mammalian central nervous system (CNS). The excitatory amino acids, including glutamate, are of great physiological importance, playing a role in a variety of physiological processes, such as long-term potentiation (learning and memory), the development of synaptic plasticity, motor control, respiration, cardiovascular regulation, and sensory perception.

Glutamate acts via at least two distinct classes of receptors. One class is composed of the ionotropic glutamate (iGlu) receptors that act as ligand-gated ionic channels. Via activation of the iGlu receptors, glutamate is thought to regulate fast neuronal transmission within the synapse of two connecting neurons in the CNS. The second general type of receptor is the G-protein or second messenger-linked “metabotropic” glutamate (mGluR) receptor. Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

The present invention relates to potentiators of mGlu receptors, in particular mGluR2 receptors. The mGluR receptors belong to the Type III G-protein coupled receptor (GPCR) superfamily. This superfamily of GPCR'sf including the calcium-sensing receptors, GABAB receptors and pheromone receptors, which are unique in that they are activated by binding of effectors to the amino-terminus portion of the receptor protein. The mGlu receptors are thought to mediate glutamate's demonstrated ability to modulate intracellular signal transduction pathways. Ozawa, Kamiya and Tsuzuski, Prog. Neurobio., 54, 581 (1998). They have been demonstrated to be localized both pre- and post-synaptically where they can regulate neurotransmitter release, either glutamate or other neurotransmitters, or modify the post-synaptic response of neurotransmitters, respectively.

At present, there are eight distinct mGlu receptors that have been positively identified, cloned, and their sequences reported. These are further subdivided based on their amino acid sequence homology, their ability to effect certain signal transduction mechanisms, and their known pharmacological properties. Ozawa, Kamiya and Tsuzuski, Prog. Neurobio., 54, 581 (1998). For instance, the Group I mGluR receptors, which include the mGluIR and mGlu5R, are known to activate phospholipase C (PLC) via Gaq-proteins thereby resulting in the increased hydrolysis of phosphoinositides and intracellular calcium mobilization. There are several compounds that are reported to activate the Group I mGlu receptors including DHPG, (R/S)-3,5-dihydroxyphenylglycine. Schoepp, Goldworthy, Johnson, Salhoff and Baker, J. Neurochem., 63, 769 (1994); Ito, et al., keurorep., 3, 1013 (1992). The Group II mGlu receptors consist of the two distinct receptors, mGluR2 and mGluR3 receptors. Both have been found to be negatively coupled to adenylate cyclase via activation of Gai-protein. These receptors can be activated by a selective compound such as 1S,2S,5R,6S-2 aminobicyclo[3.1.0]hexane-2,6-dicarboxylate. Monn, et al., J. Med. Chem., 40, 528 (1997); Schoepp, et al., Neuropharmacol., 36, 1 (1997). The Group III mGlu receptors, including mGluR4, mGluR6, mGluR7 and mGluR8, are negatively coupled to adenylate cyclase via Gai and are potently activated by L-AP4 (L-(+)-2-amino-4-phosphonobutyric acid). Schoepp, Neurochem. Int., 24, 439 (1994).

It has become increasingly clear that there is a link between modulation of excitatory amino acid receptors, including the glutamatergic system, through changes in glutamate release or alteration in postsynaptic receptor activation, and a variety of neurological and psychiatric disorders. e.g. Monaghan, Bridges and Cotman, Ann. Rev. Pharmacol. Toxicol., 29, 365-402 (1989); Schoepp and Sacann, Neurobio. Aging, 15, 261-263 (1994); Meldrum and Garthwaite, Tr. Pharmacol. Sci., 11, 379-387 (1990). The medical consequences of such glutamate dysfunction makes the abatement of these neurological processes an important therapeutic goal. Certain pyridyl potentiators of metabotropic glutamate receptors are disclosed in PCT WO 01/56990.

### SUMMARY OF THE INVENTION

The present invention is directed to compounds which are potentiators of metabotropic glutamate receptors, including the mGluR2 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### DETAILED DESCRIPTION OF THE INVENTION

The present invention is directed to compounds of the formula I: 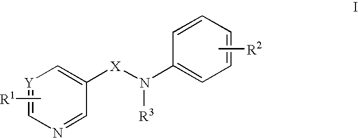
wherein:
X is selected from the group consisting of:
(1) C₁₋₆alkyl, and
(2) a bond;
   Y is N to form a pyrimidinyl ring, or
Y is C wheren R¹ at the adjacent position is joined to Y as a phenyl group to form a quinolinyl ring;
   R¹ and R² may include multiple substituents and are independently selected from the group consisting of:
(1) hydrogen,
(2) halogen,
(3) C₁₋₆alkyl, unsubstituted or substituted with:
   (a) halogen,
   (b) —CN,
   (c) —COC₁₋₆alkyl
   (d) —CO₂C₁₋₆alkyl,
   (e) phenyl, which is unsubstituted or substituted with fluoro, bromo, —C₁₋₆alkyl, —CF₃ or —O—C₁₋₆alkyl,
(4) —O—C₁₋₆alkyl, unsubstituted or substituted with halogen,
(5) —O—C₃₋₆cycloalkyl,
(6) phenyl, unsubstituted or substituted with halogen, —C₁₋₆alkyl, —CF₃ or —O—C₁₋₆alkyl,
(7) —O-phenyl, unsubstituted or substituted with halogen, —C₁₋₆alkyl, —CF₃ or —O—C₁₋₆alkyl, and
(8) hydroxyl;
   R³ is selected from the group consisting of:
(1) hydrogen,
(2) —SO₂R⁹,
   wherein R⁹ is independently selected from:
   (a) —C₁₋₆alkyl, which is unsubstituted or substituted with 1-6 halogen or hydroxyl,
   (b) —C₃₋₆cycloalkyl, which is unsubstituted or substituted with 1-6 halogen or hydroxyl,
   (c) benzyl, and
   (d) phenyl,
(3) —CO₂R¹⁰,
   wherein R¹⁰ is independently selected from:
   (a) hydrogen,
   (b) —C₁₋₆alkyl, which is unsubstituted or substituted with 1-6 fluoro,
   (c) benzyl, and
   (d) phenyl;
      and pharmaceutically acceptable salts thereof and individual diastereomers thereof.

An embodiment of the present invention includes compounds of the formula Ia: 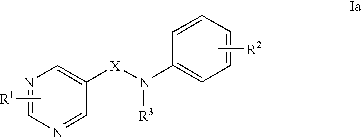
wherein R¹, R², R³, A and X are defined herein;
or a pharmaceutically acceptable salt thereof or an individual enantiomer or diastereomer thereof.

An embodiment of the present invention includes compounds of the formula Ib: 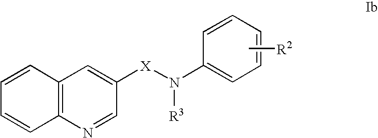
wherein R¹, R², R³, A and X are defined herein;
or a pharmaceutically acceptable salt thereof or an individual enantiomer or diastereomer thereof.

An embodiment of the present invention includes compounds wherein

X is —CH₂—.

An embodiment of the present invention includes compounds wherein

X is —CH₂CH₂—.

An embodiment of the present invention includes compounds wherein

R¹ is hydrogen.

An embodiment of the present invention includes compounds wherein R² is selected from the group consisting of:
(1) hydrogen,
(2) halogen,
(3) C₁₋₆alkyl, unsubstituted or substituted with halogen or phenyl, which is unsubstituted or substituted with fluoro, bromo, —C₁₋₆alkyl, —CF₃ or —O—C₁₋₆alkyl,
(4) —O—C₁₋₆alkyl, unsubstituted or substituted with halogen,
(5) -phenyl, unsubstituted or substituted with halogen, —C₁₋₆alkyl, —CF₃ or —O—C₁₋₆alkyl, and
(6) —O-phenyl, unsubstituted or substituted with halogen, —C₁₋₆alkyl, —CF₃ or —O—C₁₋₆alkyl.

An embodiment of the present invention includes compounds wherein R² is selected from the group consisting of:
(1) hydrogen,
(2) fluoro,
(3) bromo,
(4) C₁₋₆alkyl, unsubstituted or substituted with halogen or phenyl, which is unsubstituted or substituted with fluoro, bromo, —CH₃, —CF₃ or —O—CH₃,
(5) —O—C₁₋₆alkyl, unsubstituted or substituted with halogen,
(6) -phenyl, unsubstituted or substituted with fluoro, bromo, —CH₃, —CF₃, or —O—CH₃, and
(7) —O-phenyl, unsubstituted or substituted with fluoro, bromo, —CH₃, —CF₃, or —O—CH₃

An embodiment of the present invention includes compounds wherein R² is selected from the group consisting of:
(1) hydrogen,
(2) fluoro,
(3) bromo,
(4) -phenyl,
(5) —CH₂-phenyl,
(6) —CH₂-phenyl-CF₃,
(7) —O—CH₃,
(8) —O—CF₃,
(9) —O—CH(CH₃)CH₂CH₂CH₃,
(10) —O-phenyl,
(11) —O-phenyl-F,
(12) —O-phenyl-Br,
(13) —O-phenyl-CH₃, and
(14) —O-phenyl-O—CH₃.

An embodiment of the present invention includes compounds wherein

R³ is hydrogen.

An embodiment of the present invention includes compounds wherein

R³ is —SO₂—C₁₋₆alkyl, which is unsubstituted or substituted with fluoro.

An embodiment of the present invention includes compounds wherein

R³ is —SO₂—CH₂CF₃.

Specific embodiments of the present invention include a compound which selected from the group consisting of:
N-[3-(cyclopentyloxy)phenyl]-2,2,2-trifluoro-N-(pyrimidin-5-ylmethyl)ethanesulfonamide;
2,2,2-trifluoro-N-(pyrimidin-5-ylmethyl)-N-{3-[2-(trifluoromethyl)benzyl]phenyl}ethanesulfonamide;
2,2,2-trifluoro-N-[3-(2-methoxyphenoxy)phenyl]-N-(pyrimidin-5-ylmethyl)ethane-sulfonamide;
N-[3-({[tert-butyl(dimethyl)silyl]oxy}methyl)phenyl]-2,2,2-trifluoro-N-(pyrimidin-5-ylmethyl)ethanesulfonamide;
3-{(pyrimidin-5-ylmethyl) [(2,2,2-trifluoroethyl)sulfonyl]amino}benzyl benzoate;
[3-(1-methylbutoxy)phenyl](quinolin-3-ylmethyl)amine;
2,2,2-trifluoro-N-[3-(1-methylbutoxy)phenyl]-N-(pyrimidin-5-ylmethyl)ethanesulfonamide;
2,2,2-trifluoro-N-(pyrimidin-5-ylmethyl)-N-{3-[2-(trifluoromethyl)benzyl]phenyl}-ethanesulfonamide;
N-(pyrimidin-5-ylmethyl)-3-(trifluoromethoxy)aniline;
2,2,2-trifluoro-N-(pyrimidin-5-ylmethyl)-N-[3-(trifluoromethoxy)-phenyl]ethanesulfonamide;
{3-[(pyrimidin-5-ylmethyl)amino]-phenyl}methanol;
N-(3-ethoxyphenyl)-2,2,2-trifluoro-N-(pyrimidin-5-ylmethyl)ethane-sulfonamide;
(3-methoxyphenyl)(pyrimidin-5-ylmethyl)amine;
2,2,2-trifluoro-N-(3-methoxy-phenyl)-N-(pyrimidin-5-ylmethyl)ethanesulfonamide;
3-[(pyrimidin-5-ylmethyl)amino]-phenol;
N-[3-({[tert-butyl(dimethyl)-silyl]oxy}methyl)phenyl]-2,2,2-trifluoro-N-(pyrimidin-5-ylmethyl)ethanesulfonamide;
2,2,2-trifluoro-N-[3-(phenoxymethyl)phenyl]-N-(pyrimidin-5-ylmethyl)ethanesulfonamide;
N-[3-(cyclopentyloxy)phenyl]-2,2,2-trifluoro-N-(pyrimidin-5-ylmethyl)ethanesulfonamide;
2,2,2-trifluoro-N-[3-(hydroxymethyl)phenyl]-N-(pyrimidin-5-ylmethyl)ethanesulfonamide;
2,2,2-trifluoro-N-[3-(2-methylphenoxy)phenyl]-N-(pyrimidin-5-ylmethyl)ethanesulfonamide;
2,2,2-trifluoro-N-[3-(2-fluorophenoxy)phenyl]-N-(pyrimidin-5-ylmethyl)ethanesulfonamide;
2,2,2-trifluoro-N-(4-phenoxyphenyl)-N-(pyrimidin-5-ylmethyl)ethanesulfonamide;
2,2,2-trifluoro-N-(pyrimidin-5-ylmethyl)-N-{3-[(trifluoromethyl)-thio]phenyl}ethanesulfonamide;
3-{(pyrimidin-5-ylmethyl)[(2,2,2-trifluoroethyl)sulfonyl]amino}-benzyl benzoate;
(3-benzylphenyl)(pyrimidin-5-ylmethyl)amine;
N-[3-(2-bromophenoxy)phenyl]-2,2,2-trifluoro-N-(pyrimidin-5-ylmethyl)ethanesulfonamide;
2,2,2-trifluoro-N-[4-(1-methylbutoxy)phenyl]-N-(pyrimidin-5-ylmethyl)ethanesulfonamide;
N-(3-benzylphenyl)-2,2,2-trifluoro-N-(pyrimidin-5-ylmethyl)ethane-sulfonamide;
N-biphenyl-3-yl-2,2,2-trifluoro-N-(pyrimidin-5-ylmethyl)ethane-sulfonamide;
2,2,2-trifluoro-N-(pyrimidin-5-ylmethyl)-N-(2,2,3,3-tetrafluoro-2,3-dihydro-1,4-benzodioxin-6-yl)ethanesulfonamide;
N-(pyrimidin-5-ylmethyl)aniline;
2,2,2-trifluoro-N-[3-(2-methoxyphenoxy)phenyl]-N-(pyrimidin-5-ylmethyl)ethane-sulfonamide;
2,2,2-trifluoro-N-(3-phenoxyphenyl)-N-(pyrimidin-5-ylmethyl)ethanesulfonamide;
[3-(1-methylbutoxy)phenyl]-(quinolin-3-ylmethyl)amine;
N-(quinolin-3-ylmethyl)-3-(trifluoromethoxy)aniline; and pharmaceutically acceptable salts thereof.

The compounds of the present invention are potentiators of metabotropic glutamate (mGluR) receptor function, in particular they are potentiators of mGluR2 receptors. That is, the compounds of the present invention do not appear to bind at the glutamate recognition site on the mGluR receptor, but in the presence of glutamate or a glutamate agonist, the compounds of the present invention increase mGluR receptor response. The present potentiators are expected to have their effect at mGluR receptors by virtue of their ability to increase the response of such receptors to glutamate or glutamate agonists, enhancing the function of the receptors. It is recognized that the compounds of the present invention would be expected to increase the effectiveness of glutamate and glutamate agonists of the mGluR2 receptor. Thus, the potentiators of the present invention are expected to be useful in the treatment of various neurological and psychiatric disorders associated with glutamate dysfunction described to be treated herein and others that can be treated by such potentiators as are appreciated by those skilled in the art.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

As appreciated by those of skill in the art, halo or halogen as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C₁₋₈alkyl specifically includes methyl, ethyl, n-propyl, iso-propyl, n-butyl, iso-butyl, tert-butyl, pentyl, and hexyl. A group which is designated as being independently substituted with substituents may be independently substituted with multiple numbers of such substituents.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of Formula I are meant to also include the pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

The subject compounds are useful in a method of potentiating metabotorpic glutamate receptor activity in a patient such as a mammal in need of such inhibition comprising the administration of an effective amount of the compound. The present invention is directed to the use of the compounds disclosed herein as potentiators of metabotorpic glutamate receptor activity. In addition to primates, especially humans, a variety of other mammals can be treated according to the method of the present invention.

The present invention is further directed to a method for the manufacture of a medicament for potentiating metabotorpic glutamate receptor activity in humans and animals comprising combining a compound of the present invention with a pharmaceutical carrier or diluent.

The subject treated in the present methods is generally a mammal, preferably a human being, male or female, in whom potentiation of metabotorpic glutamate receptor activity is desired. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with the disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of metabotropic glutamate receptor activity, in particular mGluR2 activity, may be demonstrated by methodology known in the art. Inhibition constants are determined as follows. The compounds of the present invention were tested in a [³⁵S]-GTPγS assay. The stimulation of [³⁵S]-GTPγS binding is a common functional assay to monitor Gαi-coupled receptor in native and recombinant receptor membrane preparation. Membrane from cells stably expressing hmGlu2 CHO-K1 (50 μg) were incubated in a 96 well plate for 1 hour in the presence of GTPγS³⁵ (0.05 nM), GDP (5 μM) and compounds. The reaction was stopped by rapid filtration over Unifilter GF/B plate (Packard, Bioscience, Meriden Conn.) using a 96-well cell harvester (Brandel Gaithersburg, Md.). The filter plates were counted using Topcount counter (Packard, Bioscience, Meriden Conn., USA). When compounds were evaluated as potentiator they were tested in the presence of glutamate (11 M). The activation (agonist) or the potentiation of glutamate (potentiator) curves were fitted with a four parameters logistic equation giving EC₅₀ and Hill coefficient using the iterative non linear curve fitting software GraphPad (San Diego Calif., USA).

In particular, the compounds of the following examples had activity in potentiating the mGluR2 receptor in the aforementioned assays, generally with an EC₅₀ of less than about 10 μM. Preferred compounds within the present invention had activity in potentiating the mGluR2 receptor in the aforementioned assays with an EC₅₀ of less than about 1 μM. Such a result is indicative of the intrinsic activity of the compounds in use as potentiators of mGluR2 receptor activity.

Metabotropic glutamate receptors including the mGluR2 receptor have been implicated in a wide range of biological functions. This has suggested a potential role for these receptors in a variety of disease processes in humans or other species.

The compounds of the present invention have utility in treating, preventing, ameliorating, controlling or reducing the risk of a variety of neurological and psychiatric disorders associated with glutamate dysfunction, including one or more of the following conditions or diseases: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder.

Of the disorders above, the treatment of migraine, anxiety, schizophrenia, and epilepsy are of particular importance. In a preferred embodiment the present invention provides a method for treating migraine, comprising:

administering to a patient in need thereof an effective amount of a compound of formula I. In another preferred embodiment the present invention provides a method for preventing or treating anxiety, comprising: administering to a patient in need thereof an effective amount of a compound of formula I. Particularly preferred anxiety disorders are generalized anxiety disorder, panic disorder, and obsessive compulsive disorder. In another preferred embodiment the present invention provides a method for treating schizophrenia, comprising: administering to a patient in need thereof an effective amount of a compound of formula I. In yet another preferred embodiment the present invention provides a method for treating epilepsy, comprising: administering to a patient in need thereof an effective amount of a compound of formula I.

Of the neurological and psychiatric disorders associated with glutamate dysfunction which are treated according to the present invention, the treatment of migraine, anxiety, schizophrenia, and epilepsy are particularly preferred. Particularly preferred anxiety disorders are generalized anxiety disorder, panic disorder, and obsessive compulsive disorder.

Thus, in a preferred embodiment the present invention provides a method for treating migraine, comprising: administering to a patient in need thereof an effective amount of a compound of formula I or a pharmaceutical composition thereof. In one of the available sources of diagnostic tools, Dorland's Medical Dictionary (23'd Ed., 1982, W.B. Saunders Company, Philidelphia, Pa.), migraine is defined as a symptom complex of periodic headaches, usually temporal and unilateral, often with irritability, nausea, vomiting, constipation or diarrhea, and photophobia. As used herein the term “migraine” includes these periodic headaches, both temporal and unilateral, the associated irritability, nausea, vomiting, constipation or diarrhea, photophobia, and other associated symptoms. The skilled artisan will recognize that there are alternative nomenclatures, nosologies, and classification systems for neurological and psychiatric disorders, including migraine, and that these systems evolve with medical scientific progress.

In another preferred embodiment the present invention provides a method for treating anxiety, comprising: administering to a patient in need thereof an effective amount of a compound of formula I or a pharmaceutical composition thereof. At present, the fourth edition of the Diagnostic and Statistical Manual of Mental Disorders (DSM-IV) (1994, American Psychiatric Association, Washington, D.C.), provides a diagnostic tool including anxiety and related disorders. These include: panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to a general medical condition, substance-induced anxiety disorder and anxiety disorder not otherwise specified. As used herein the term “anxiety” includes treatment of those anxiety disorders and related disorder as described in the DSM-IV. The skilled artisan will recognize that there are alternative nomenclatures, nosologies, and classification systems for neurological and psychiatric disorders, and particular anxiety, and that these systems evolve with medical scientific progress. Thus, the term “anxiety” is intended to include like disorders that are described in other diagnostic sources.

In another preferred embodiment the present invention provides a method for treating depression, comprising: administering to a patient in need thereof an effective amount of a compound of formula I or a pharmaceutical composition thereof. At present, the fourth edition of the Diagnostic and Statistical Manual of Mental Disorders (DSM-IV) (1994, American Psychiatric Association, Washington, D.C.), provides a diagnostic tool including depression and related disorders. Depressive disorders include, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder; or bipolar disorders or manic depression, for example, bipolar I disorder, bipolar II disorder and cyclothymic disorder. As used herein the term “depression” includes treatment of those depression disorders and related disorder as described in the DSM-IV.

In another preferred embodiment the present invention provides a method for treating epilepsy, comprising: administering to a patient in need thereof an effective amount of a compound of formula I or a pharmaceutical composition thereof. At present, there are several types and subtypes of seizures associated with epilepsy, including idiopathic, symptomatic, and cryptogenic. These epileptic seizures can be focal (partial) or generalized. They can also be simple or complex. Epilepsy is described in the art, such as Epilepsy: A comprehensive textbook. Ed. by Jerome Engel, Jr. and Timothy A. Pedley. (Lippincott-Raven, Philadelphia, 1997). At present, the International Classification of Diseases, Ninth Revision, (ICD-9) provides a diagnostic tool including epilepsy and related disorders. These include: generalized nonconvulsive epilepsy, generalized convulsive epilepsy, petit mal status epilepticus, grand mal status epilepticus, partial epilepsy with impairment of consciousness, partial epilepsy without impairment of consciousness, infantile spasms, epilepsy partialis continua, other forms of epilepsy, epilepsy, unspecified, NOS. As used herein the term “epilepsy” includes these all types and subtypes. The skilled artisan will recognize that there are alternative nomenclatures, nosologies, and classification systems for neurological and psychiatric disorders, including epilepsy, and that these systems evolve with medical scientific progress.

The subject compounds are further useful in a method for the prevention, treatment, control, amelioration, or reducation of risk of the diseases, disorders and conditions noted herein.

The subject compounds are further useful in a method for the prevention, treatment, control, amelioration, or reduction of risk of the aforementioned diseases, disorders and conditions in combination with other agents, including an mGluR agonist.

The term “potentiated amount” refers to an amount of an mGluR agonist, that is, the dosage of agonist which is effective in treating the neurological and psychiatric disorders described herein when administered in combination with an effective amount of a compound of the present invention. A potentiated amount is expected to be less than the amount that is required to provided the same effect when the mGluR agonist is administered without an effective amount of a compound of the present invention.

A potentiated amount can be readily determined by the attending diagnostician, as one skilled in the art, by the use of conventional techniques and by observing results obtained under analogous circumstances. In determining a potentiated amount, the dose of an mGluR agonist to be administered in combination with a compound of formula I, a number of factors are considered by the attending diagnostician, including, but not limited to: the mGluR agonist selected to be administered, including its potency and selectivity; the compound of formula I to be coadministered; the species of mammal; its size, age, and general health; the specific disorder involved; the degree of involvement or the severity of the disorder; the response of the individual patient; the modes of administration; the bioavailability characteristics of the preparations administered; the dose regimens selected; the use of other concomitant medication; and other relevant circumstances.

A potentiated amount of an mGluR agonist to be administered in combination with an effective amount of a compound of formula I is expected to vary from about 0.1 milligram per kilogram of body weight per day (mg/kg/day) to about 100 mg/kg/day and is expected to be less than the amount that is required to provided the same effect when administered without an effective amount of a compound of formula I. Preferred amounts of a co-administered mGlu agonist are able to be determined by one skilled in the art.

The compounds of the present invention may be used in combination with one or more other drugs in the treatment, prevention, control, amelioration, or reduction of risk of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also includes therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

The above combinations include combinations of a compound of the present invention not only with one other active compound, but also with two or more other active compounds.

Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The pharmaceutical composition and method of the present invention may further comprise other therapeutically active compounds as noted herein which are usually applied in the treatment of the above mentioned pathological conditions.

In the treatment, prevention, control, amelioration, or reduction of risk of conditions which require potentiation of metabotorpic glutamate receptor activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

When treating, preventing, controlling, ameliorating, or reducing the risk of neurological and psychiatric disorders associated with glutamate dysfunction or other diseases for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made according to procedures known in the art or as illustrated herein. The compounds of the present invention can be prepared in a variety of fashions.

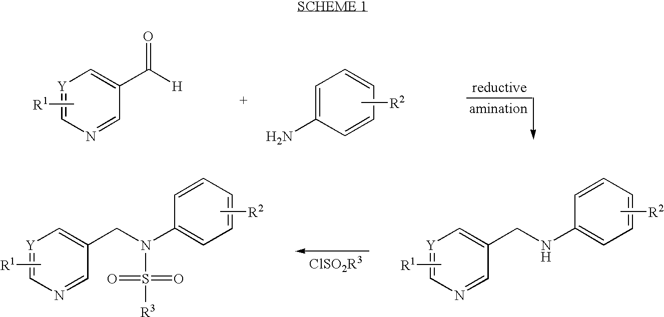

An appropriately substituted pyrimidine (or quinoline) can be prepared via reductive amination followed by sulfonylation as illustrated in Scheme 1. A substituted pyrimidine aldehyde (either purchased commercially or prepared using techniques well known in the art) is reacted with a substituted aniline in the presence of a reducing agent, such as sodium borohydride or sodium triacetoxyborohydride, can be used in solvents such as methanol or dichloroethane. The reaction generally proceeds either by refluxing the reagents in methanol then adding sodium borohydride at 0° C. and allowing the reaction to warm to ambient temperature over a period of several hours, or by adding sodium triacetoxyboro-hydride to the reagents at ambient temperature and then maintaining ambient temperature for several more hours. The product from the reaction can be isolated and purified employing standard techniques such as solvent extraction, chromatography, crystallization, distillation and the like.

The product obtained is then sulfonylated with variously substituted sulfonyl chloride compounds. These sulfonyl chloride compounds are reacted in the presence of a base (potassium carbonate, pyridine, and the like) in a suitable solvent (dichloroethane, pyridine, etc). The reaction is generally run at ambient temperature for several hours. The product from the reaction can be isolated and purified employing standard techniques such as solvent extraction, chromatography, crystallization, distillation and the like.

In some cases the final product may be further modified, for example, by manipulation of substituents. These manipulations may include, but are not limited to, reduction, oxidation, alkylation, acylation, and hydrolysis reactions which are commonly known to those skilled in the art.

In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### EXAMPLE 1

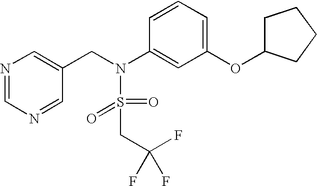

### N-[3-(cyclopentyloxy)phenyl]-2,2,2-trifluoro-N-(pyrimidin-5-ylmethyl)ethanesulfonamide

Diisopropyl azodicarboxylate (0.45 mL, 2.3 mmol) was added dropwise to a stirred solution of 3-aminophenol (250 mg, 2.3 mmol), cyclopentanol (0.25 mL, 2.8 mmol), and triphenylphosphine (600 mg, 2.3 mmol) in tetrahydrofuran (1.4 mL) at 0° C. After stirring for an additional 12 hr at room temperature, the reaction mixture was rotovapped in vacuo to remove volatile solvents. The residue was directly purified via column chromatography on silica gel (eluting 5-50% ethyl acetate/hexanes) to give 157 mg (40%) of [3-(cyclopentyloxy)phenyl]amine as red oil. To a solution of [3-(cyclopentyloxy)phenyl]-amine (157 mg, 0.89 mmol), pyrimidine aldehyde (96 mg, 0.89 mmol) and acetic acid (51 uL, 0.89 mmol) in dichloroethane 3.6 mL) was added sodium triacetoxyborohydride (283 mg, 1.3 mmol) at room temperature. The resulting mixture was stirred for an additional 2 hr or until TLC indicated complete conversion. The reaction mixture was then rotovapped in vacuo to remove volatile solvents. The residue was directly purified via column chromatography on silica gel (eluting 15-100% ethyl acetate/hexanes) to give 200 mg (83%) of [3-(cyclopentyloxy)phenyl]-(pyrimidin-5-ylmethyl)amine as brown oil.

A solution of 2,2,2-trifluoroethanesulfonyl chloride (0.11 mL, 1.0 mmol) was added dropwise to a solution of [3-(cyclopentyloxy)phenyl](pyrimidin-5-ylmethyl)amine (200 mg, 0.74 mmol) in dichloroethane (2.8 mL) and pyridine (1.4 mL) at 0° C. The reaction mixture was allowed to gradually warm to room temperature and then stirred at room temperature until TLC indicated completion of reaction. The solution was then rotovapped in vacuo to remove volatile solvents. The residue was directly purified via column chromatography on silica gel (eluting with 0-100% ethyl acetate/dichloromethane) to give N-[3-(cyclopentyloxy)phenyl]-2,2,2-trifluoro-N-(pyrimidin-5-ylmethyl)ethanesulfonamide as a solid. ¹H NMR (CDCl₃, 500 MHz), δ 9.14 (s, 1H), 8.61 (s, 2H), 7.29-7.25 (m, 1H), 6.87-6.85 (m, 1H), 6.81-6.79 (m, 1H), 6.72-6.71 (m, 1H), 4.89 (s, 2H), 4.67-4.65 (m, 1H), 3.88-3.83 (m, 2H), 1.90-1.61 (m, 8H). MS (ESI): 416 (M⁺+H).

### EXAMPLE 2

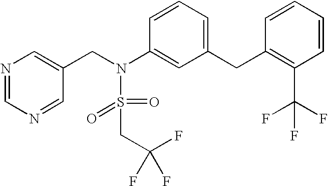

### 2,2,2-trifluoro-N-(pyrimidin-5-ylmethyl)-N-{3-[2-(trifluoromethyl)benzyl]phenyl}ethanesulfonamide

A heterogeneous solution of 1-(bromomethyl)-2-(trifluoromethyl)benzene (1.0 g, 4.2 mmol) and 3-nitrophenyl boronic acid (1.1 g, 6.3 mmol) in dimethoxyethane (38 mL) and 2M solution of sodium carbonate (12.7 mL, 25.5 mmol) was purged with nitrogen for 5 min. Palladium triphenylphosphine (155 mg, 0.13 mmol) was then added to the biphasic mixture. The reaction mixture was heated to 50° C. for 16 hr, cooled to room temperature, and quenched by addition of water and ethyl acetate. The organic layer was separated, dried over Na₂SO₄ and concentrated in vacuo. The residue was purified via column chromatography on silica gel (eluting 15-80% ethyl acetate/hexanes) to give 1.16 g (98%) of 1-(3-nitrobenzyl)-2-(trifluoromethyl)benzene as a clear, colorless oil. Hydrazine monohydrate (2 mL, 41.2 mmol) and a catalytic amount of palladium on carbon was added to a solution of 1-(3-nitrobenzyl)-2-(trifluoromethyl)benzene (1.16 g, 4.1 mmol) in ethyl alcohol (200 mL) at ambient temperature. The resulting mixture was heated to reflux for 16 hr. The reaction was cooled to room temperature and filtered over Celite. The filtrate was concentrated in vacuo to give 0.95 g (92%) of 3-[2-(trifluoromethyl)benzyl]aniline.

A solution of pyrimidine aldehyde (230 mg, 2.1 mmol) and 3-[2-(trifluoromethyl)benzyl]aniline (450 mg, 1.8 mmol) in methanol (4.5 mL) was refluxed for 2 hr. The resulting mixture was cooled to 0° C. and then allowed to warm back to room temperature after the addition of sodium borohydride (170 mg, 4.5 mmol). After stirring for an additional 16 h at room temperature, the mixture was rotovapped in vacuo to remove volatile solvents. The residue was directly purified by silica gel chromatography (eluting first with 20-100% ethyl acetate/hexanes followed by 0-20% MeOH/dichloromethane) to give 141 mg of N-(pyrimidin-5-ylmethyl)-3-[2-(trifluoromethyl)-benzyl]aniline. A neat solution of 2,2,2-trifluoroethanesulfonyl chloride (64 uL, 0.58 mmol) was added dropwise to a solution of N-(pyrimidin-5-ylmethyl)-3-[2-(trifluoromethyl)benzyl]aniline (141 mg, 0.41 mmol) in dichloroethane (1.6 mL) and pyridine (0.8 mL) at 0° C. The reaction mixture was allowed to gradually warm to room temperature and stirred at room temperature until TLC indicated completion of reaction. Volatile solvents were removed using a rotary evaporator. The residue was directly purified via column chromatography on silica gel (eluting with 0-100% ethyl acetate/dichloromethane) to give 2,2,2-trifluoro-N-(pyrimidin-5-ylmethyl)-N-{3-[2-(trifluoromethyl)benzyl]phenyl}ethanesulfonamide as solid.

¹H NMR (CDCl₃, 500 MHz), δ 9.12 (s, 1H), 8.55 (s, 2H), 7.70-7.68 (d, 1H), 7.48-7.47 (m, 1H), 7.39-7.29 (m, 3H), 7.18-7.17 (d, 1H), 7.12-7.09 (m, 1H), 6.94 (s, 1H), 4.85 (s, 2H), 4.16 (s, 2H), 3.82-3.77 (m, 2H).

MS (ESI): 490 (M⁺+H).

### COMPOUND 1

### [3-(2-methoxyphenoxy)phenyl]amine

To a solution of 1-fluoro-3-nitrobenzene (0.70 g, 4.96 mmol), guaiacol (1.2 g, 9.9 mmol) in anhydrous DMF (10 mL) was added K₂CO₃ (1.4 g, 9.9 mmol). The resulting mixture was heated to 110° C. for 16 h then allowed to cool to ambient temperature. The reaction was diluted with EtOAc (100 mL), washed with H₂O (200 mL), and then brine (200 mL). The organic layer was dried over MgSO₄, filtered and concentrated in vacuo. The residue was purified by silica gel chromatography eluting with hexane:EtOAc (3:7) to afford 1-methoxy-2-(3-nitrophenoxy)benzene as a yellow oil. ¹H NMR (CDCl₃, 300 MHz) δ 7.84-7.86 (d, 1H), 7.66-7.67 (d, 1H), 7.39-7.43 (t, 1H), 7.22-7.24 (m, 2H), 7.03-7.08 (m, 2H), 6.98-6.99 (t, 1H), 3.7 (s, 3H). Iron powder (0.48 g, 8.4 mmol) and concentrated HCl solution (0.5 mL) were added to a solution of 1-methoxy-2-(3-nitrophenoxy)benzene (0.35 g, 1.4 mmol) in ethanol. After heating the resulting heterogeneous mixture to 55° C. for 2 h, the reaction was filtered through Celite. The filtrate was concentrated in vacuo and the residue was diluted with EtOAc (10 mL), washed with H₂O (100 mL), then brine (100 mL). The organic layer was dried over MgSO₄, filtered and concentrated in vacuo to afford pure [3-(2-methoxyphenoxy)phenyl]amine as a clear oil. ¹H NMR (CDCl₃, 300 MHz) δ 7.30-7.11 (m, 1H), 7.03-7.06 (t, 1H), 6.98-7.00 (d, 2H), 6.90-6.92 (t, 1H), 6.26-6.34 (m, 2H), 6.27 (s, 1H), 3.8 (s, 3H), 3.46 (S, 2H).

### EXAMPLE 3

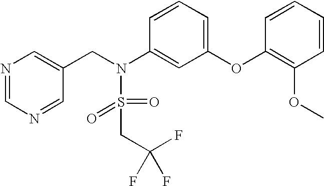

### 2,2,2-trifluoro-N-[3-(2-methoxyphenoxy)phenyl]-N-(pyrimidin-5-ylmethyl)ethane-sulfonamide

To a solution of [3-(2-methoxyphenoxy)phenyl]amine (0.30 g, 1.39 mmol), pyrimidine-5-carbaldehyde (0.16 g, 1.5 mmol) in dichloroethane (3 ml) was added acetic acid (0.08, 1.4 mmol) and sodiumtriacetoxyborohydride (0.50 g, 2.1 mmol) at ambient temperature. After stirring for 2 h, the reaction was quenched with EtOAc (100 mL), washed with saturated sodium hydrogencarbonate solution (50 ml), H₂O (50 mL), then brine (SOnL). The organic layer was dried over MgSO₄, filtered and concentrated in vacuo. The residue was purified by silica gel chromatography eluting with EtOAc to afford [3-(2-methoxyphenoxy)phenyl](pyrimidin-5-ylmethyl)amine as a yellow solid. ¹H NMR (CDCl₃, 300 MHz) 9.12 (s, 1H), 8.67 (s, 2H), 5.97-7.11 (m, 2H), 6.95-6.98 (m, 2H), 6.89-6.91 (m, 1H), 6.28-6.31 (m, 2H), 5.19 (s, 1H), 4.29-4.31 (m, 2H), 4.23 (s, 1H), 3.79 (s, 3H). [3-(2-methoxyphenoxy)phenyl](pyrimidin-5-ylmethyl)amine (0.05 g, 0.16 mmol) was dissolved in dichloroethane (4 mL) and pyridine (2 ml) and cooled to −5° C. under argon. A neat solution of 2,2,2-trifluoroethylsulfonyl chloride (0.04 g, 0.21 mmol) was added dropwise due to exotherm. After 30 min stirring at 0° C., the reaction mixture was stirred at ambient temperature for an additional 16 h before quenching with potassium carbonate (0.10 g, 0.69 mmol). The mixture was diluted with EtOAc (50 mL), washed with H₂O (50 mL), then brine (50 mL). The organic layer was dried over MgSO₄, filtered and concentrated in vacuo. The residue was purified by Prep TLC eluting with hexane:EtOAc (1:9) to afford 2,2,2-trifluoro-N-[3-(2-methoxyphenoxy)phenyl]-N-(pyrimidin-5-ylmethyl)ethanesulfonamide. ¹H NMR (CDCl₃, 300 MHz) δ 9.13(s, 1H), 8.57 (s, 2H), 7.27 (t, 1H), 7.19 (m, 1H0, 7.00(d, 1H), 6.95 (m, 2H), 6.90 (d, 2H), 6.79 (s, 1H), 4.85 (s, 2H), 3.82 (m, 5H). MS 454.2 (M⁺+H).

### EXAMPLE 4

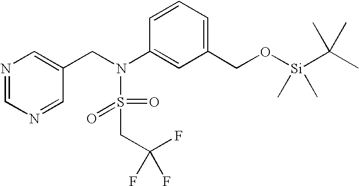

### N-[3-({[tert-butyl(dimethyl)silyl]oxy}methyl)phenyl]-2,2,2-trifluoro-N-(pyrimidin-5-ylmethyl)ethanesulfonamide

To a solution of the {3-[(pyrimidin-5-ylmethyl)amino]phenyl}methanol (600 mg, 2.79 mmol) in dimethylformamide (10 mL) was added t-butyldimethylsilyl chloride (502 mg, 3.34 mmol) and Hunig's Base (504 mg, 3.91 mmol). The solution was stirred for 12 h at ambient temperature. Analysis by TLC indicated that the reaction was done (product rf=0.44 in 66% ethyl acetate/hexanes). The solution was diluted with ethyl acetate (25 mL) and water (10 mL). The organic phase was separated and the aqueous phase was washed with ethyl acetate (25 mL). The organic phases were combined, dried with sodium sulfate and concentrated using a rotary evaporator. The crude compound was then treated with dichloroethane (10 mL), pyridine (440 mg, 5.58 mmol) and 2,2,2-trifluoroethanesulfonyl chloride (559 mg, 3.07 mmol) at ambient temperature. After stirring for 5 h, TLC indicated that the reaction was complete (product rf=0.30, 50% ethyl acetate/hexanes). The reaction was quenched by adding water (15 mL) and extracted twice with ethyl acetate (2×25 mL). The organic phases were dried with sodium sulfate and concentrated using a rotary evaporator. The sulfonamide was purified by automated silica gel flash chromatography eluting with ethyl acetate/hexanes (starting with 10% ethyl acetate in hexanes and ending with 80% ethyl acetate in hexanes). This produced N-[3-(f{[tert-butyl(dimethyl)silyl]oxy}-methyl)phenyl]-2,2,2-trifluoro-N-(pyrimidin-5-ylmethyl)ethanesulfonamide. ¹H NMR (DMSO-d₆, 500 MHz), o 9.02 (s, 1H), 8.50 (s, 2H), 7.26-7.20 (m, 3H), 7.01 (d, 1H), 4.82 (s, 2H), 4.63 (s, 2H), 3.75 (q, 2H), 0.85 (s, 9H), 0.00 (s, 6H). MS (ESI) 476.1 (M⁺+H).

### EXAMPLE 5

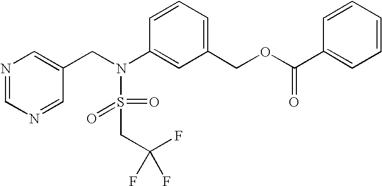

### 3-{(pyrimidin-5-ylmethyl)[(2,2,2-trifluoroethyl)sulfonyl]amino}benzyl benzoate

Acetic acid (131 mg, 2.19 mmol) and tetrabutylammonium fluoride (1.0 M in tetrahydrofuran, 2.19 mL, 2.19 mmol) were added to a solution of N-[3-({[tert-butyl(dimethyl)silyl]oxy}methyl)phenyl]-2,2,2-trifluoro-N-(pyrimidin-5-ylmethyl)ethanesulfonamide (520 mg, 1.09 mmol) in tetrahydrofuran (10 mL) at ambient temperature. After stirring 30 min, the reaction was determined to be complete by TLC analysis (product rf=0.22, 50% ethyl acetate/hexanes). The crude reaction mixture was concentrated using a rotary evaporator and directly purified by automated silica gel flash chromatography (eluting with a gradient starting with 20% ethyl acetate hexanes and ending with 100%) to yield 430 mg of the desired alcohol. The alcohol (106 mg, 0.33 mmol) was then dissolved in dichloromethane (10 mL) and treated with Hunig's base (64 mg, 0.49 mmol) and benzoyl chloride (60 mg, 0.43 mmol) at ambient temperature. After stirring for 3 hours, TLC analysis indicated that the reaction was complete (product rf=0.25, 50% ethyl acetate/hexanes). The reaction was quenched with the addition of water (15 mL) and extracted with ethyl acetate (2×25 mL). After drying with sodium sulfate, the crude compound was concetrated using a rotary evaporator and purified by automated silica gel flash chromatography (eluting with a gradient starting with 15% ethyl acetate/hexanes and ending with 100%) to yield 55 mg of 3-{(pyrimidin-5-ylmethyl)[(2,2,2-trifluoroethyl)sulfonyl]amino}benzyl benzoate. ¹H NMR (DMSO-d₆, 500 MHz), δ 9.09 (s, 1H), 8.62 (s, 2H), 8.09-8.02 (m, 2H), 7.57 (m, 1H), 7.46-7.37 (m, 5H), 7.22 (m, 1H), 5.32 (s, 2H), 4.92 (s, 2H), 3.86 (q, 2H). MS (ESI) 466.1 (M⁺+H).

### EXAMPLE 6

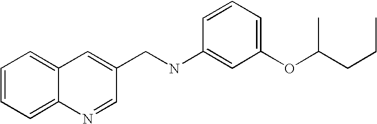

### [3-(1-methylbutoxy)phenyl](quinolin-3-ylmethyl)amine

[3-(1-methylbutoxy)phenyl]amine (0.40 g, 2.26 mmol) and quinoline-3-carbaldehyde (0.46 g, 2.26 mmol) were dissolved in anhydrous methanol (10 mL) and heated at reflux for 2 h. The resulting mixture was cooled to −5° C. under argon and solid sodium borohydride (0.18 g, 4.7 mmol) was added in one portion. After stirring for an additional 16 h at ambient temperature, the reaction mixture was concentrated in vacuo. The resulting residue was dissolved in EtOAc (100 mL), the EtOAc solution washed with H₂O (100 mL), then brine (100 mL), dried (MgSO₄), filtered and concentrated in vacuo. The residue was purified by silica gel chromatography eluting with hexane:EtOAc (6:4) to afford [3-(1-methylbutoxy)phenyl](quinolin-3-ylmethyl)amine as a brown oil. ¹H NMR (CDCl₃, 500 MHz) δ 8.90(s, 1H), 8.08-8.10 (d, 2H), 7.74-7.76 (m, 1H), 7.67-7.68 (t, 1H), 7.50-7.51 (t, 1H), 7.04-7.07 (t, 1H), 6.21-6.30 (m, 3H), 4.49 (s, 2H), 4.19-4.31 (m, 1H), 4.25 (s, 1H), 1.66-1.71 (m, 1H), 1.41-1.50 (m, 3H), 1.23-1.24 (d, 3H), 0.87-0.90 (m, 3H).

The compounds in Table 1 were synthesized as shown in Reaction Scheme I, but substituting the appropriately substituted reagent as described in the foregoing examples. The requisite starting materials were commercially available, described in the literature or readily synthesized by one skilled in the art of organic synthesis without undue experimentation.

TABLE 1

| Compound | ¹H NMR(CDCl₃, 500 MHz) δ | MS(ESI) |
|---|---|---|
| 2,2,2-trifluoro-N-[3-(1- | 9.14(s, 1H), 8.62(s, 2H), 7.30-7.26(m, | NA |
| methylbutoxy)phenyl]-N- | 1H), 6.89-6.87(m, 1H), 6.82-6.80(m, 1H), | |
| (pyrimidin-5- | 6.74-6.73(m, 1H), 4.90(s, 2H), 4.31-4.27(m, 1H), | |
| ylmethyl)ethanesulfonamide | 3.89-3.83(m, 2H), 1.68-1.24(m, 7H), 0.95-0.92(m, 3H) | |
| 2,2,2-trifluoro-N-(pyrimidin-5- | 9.12(s, 1H), 8.55(s, 2H), 7.70-7.68(d, 1H), | MS 490.1 |
| ylmethyl)-N-{3-[2- | 7.48-7.47(m, 1H), 7.39-7.29(m, 3H), 7.18- | (M⁺ + H) |
| (trifluoromethyl)benzyl]phenyl}- | 7.17(d, 1H), 7.12-7.09(m, 1H), 6.94(s, 1H), | |
| ethanesulfonamide | 4.85(s, 2H), 4.16(s, 2H), 3.82-3.77(m, 2H) | |
| N-(pyrimidin-5-ylmethyl)-3- | 9.17(s, 1H), 8.76(s, 2H), 7.17(t, 1H), 6.25(m, 1H), | MS 270.0 |
| (trifluoromethoxy)aniline | 6.52(m, 1H), 6.45(m, 1H), 4.39(s, 2H). | (M⁺ + H) |
| 2,2,2-trifluoro-N-(pyrimidin-5- | 9.15(s, 1H), 8.60(s, 2H), 7.45(t, 1H), 7.25(m, 1H), | MS 416.0 |
| ylmethyl)-N-[3-(trifluoromethoxy)- | 7.22(m, 1H), 7.13(m, 1H), 4.92(s, 2H), 3.84(q, 2H). | (M⁺ + H) |
| phenyl]ethanesulfonamide | | |
| {3-[(pyrimidin-5-ylmethyl)amino]- | 8.94(s, 1H), 8.57(t, 2H), 7.08(d, 1H), 6.65(s, 1H), | MS 216.0 |
| phenyl}methanol | 6.44(d, 1H), 4.54(br, 1H), 4.52(s, 2H), | (M⁺ + H) |
| | 4.23(m, 2H), 4.08(br, 1H) | |
| N-(3-ethoxyphenyl)-2,2,2-trifluoro- | 9.12(s, 1H), 8.60(s, 2H), 7.28(t, 1H), 6.89(m, 1H), | MS 376.0 |
| N-(pyrimidin-5-ylmethyl)ethane- | 6.83(m, 1H), 6.75(m, 1H), 4.89(s, 2H), | (M⁺ + H) |
| sulfonamide | 3.95(q, 2H), 3.83(q, 2H), 1.39(t, 3H) | |
| (3-methoxyphenyl)(pyrimidin-5- | 9.15(s, 1H), 8.75(s, 2H), 7.01(t, 1H), 6.33(m, 1H), | MS 216.1 |
| ylmethyl)amine | 6.24(m, 1H), 6.17(m, 1H), 4.37(d, 2H), | (M⁺ + H). |
| | 4.12(br, 1H), 3.75(s, 3H) | |
| 2,2,2-trifluoro-N-(3-methoxy- | 9.13(s, 1H), 8.61(s, 2H), 7.30(t, 1H), 6.91(m, 1H), | MS 362.0 |
| phenyl)-N-(pyrimidin-5- | 6.85(m, 1H), 6.76(m, 1H), 4.90(s, 2H), | (M⁺ + H) |
| ylmethyl)ethanesulfonamide | 3.83(q, 2H), 3.75(s, 3H) | |
| 3-[(pyrimidin-5-ylmethyl)amino]- | 9.04(s, 1H), 8.80(s, 2H), 7.97(s, 1H), 6.92(t, 1H), | NA |
| phenol | 6.21-6.14(m, 3H), 5.60(br, 1H).4.43(s, 2H) | |
| N-[3-({[tert-butyl(dimethyl)- | 9.02(s, 1H), 8.50(s, 2H), 7.26-7.20(m, 3H), | MS 476.1 |
| silyl]oxy}methyl)phenyl]-2,2,2- | 7.01(d, 1H), 4.82(s, 2H), 4.63(s, 2H), | (M⁺ + H) |
| trifluoro-N-(pyrimidin-5- | 3.75(q, 2H), 0.85(s, 9H), 0.00(s, 6H) | |
| ylmethyl)ethanesulfonamide | | |
| 2,2,2-trifluoro-N-[3- | 9.13(s, 1H), 8.58(s, 2H), 7.44-7.40(m, 2H), | MS 438.1 |
| (phenoxymethyl)phenyl]-N- | 7.35-7.29(m, 3H), 7.20-7.18(m, 1H), | (M⁺ + H) |
| (pyrimidin-5-ylmethyl)ethane- | 7.01-7.00(m, 1H), 6.94-6.92(m, 2H), 5.08(s, 2H), | |
| sulfonamide | 4.90(s, 2H), 3.83-8.77(m, 2H) | |
| N-[3-(cyclopentyloxy)phenyl]- | 9.14(s, 1H), 8.61(s, 2H), 7.29-7.25(m, 1H), | MS 416.1 |
| 2,2,2-trifluoro-N-(pyrimidin-5- | 6.87-6.85(m, 1H), 6.81-6.79(m, 1H), | (M⁺ + H). |
| ylmethyl)ethanesulfonamide | 6.72-6.71(m, 1H), 4.89(s, 2H), 4.67-4.65(m, 1H), | |
| | 3.88-3.83(m, 2H), 1.90-1.61(m, 8H) | |
| 2,2,2-trifluoro-N-[3- | 9.03(s, 1H), 8.56(s, 2H), 7.37-7.32(m, 2H), | MS 362.0 |
| (hydroxymethyl)phenyl]-N- | 7.29(m, 1H), 7.14(d, 1H), 4.90(s, 2H), | (M⁺ + H) |
| (pyrimidin-5-ylmethyl)ethane- | 4.63(s, 2H), 3.87(q, 2H), 3.32(br, 1H) | |
| sulfonamide | | |
| 2,2,2-trifluoro-N-[3-(2- | 9.06(s, 1H), 8.63(s, 2H), 7.38-7.35(m, 1H), | MS 438.1 |
| methylphenoxy)phenyl]-N- | 7.28-7.27(m, 1H), 7.21-7.18(m, 1H), | (M⁺ + H) |
| (pyrimidin-5-ylmethyl)ethane- | 7.13-7.09(m, 2H), 6.89-6.87(m, 1H), | |
| sulfonamide | 6.82-6.80(m, 1H), 6.72-6.71(m, 1H), 4.97(s, 2H), | |
| | 4.40-4.34(m, 2H), 2.10(s, 3H) | |
| 2,2,2-trifluoro-N-[3-(2- | 9.08(s, 1H), 8.66(s, 2H), 7.42-7.38(m, 1H), | MS 442.1 |
| fluorophenoxy)phenyl]-N- | 7.26-7.23(m, 3H), 7.15-7.13(m, 1H), | (M⁺ + H) |
| (pyrimidin-5-ylmethyl)ethane- | 7.09-7.08(m, 1H), 6.98-6.95(m, 1H), | |
| sulfonamide | 6.86-6.85(m, 1H), 4.99(s, 2H), 4.41-4.35(m, 2H) | |
| 2,2,2-trifluoro-N-(4- | 8.90(s, 1H), 8.57(s, 2H), 7.28(m, 4H), | MS 424.1 |
| phenoxyphenyl)-N-(pyrimidin-5- | 7.16(m, 1H), 6.92(m, 2H), 6.45(m, 2H), | (M⁺ + H) |
| ylmethyl)ethanesulfonamide | 4.95(s, 2H), 4.40(q, 2H). | |
| 2,2,2-trifluoro-N-(pyrimidin-5- | 9.14(s, 1H), 8.60(s, 2H), 7.67(d, 1H), | MS 432.0 |
| ylmethyl)-N-{3-[(trifluoromethyl)- | 7.57(m, 1H), 7.47(t, 1H), 7.39(m, 1H), | (M⁺ + H) |
| thio]phenyl}ethanesulfonamide | 4.93(s, 2H), 3.84(q, 2H). | |
| 3-{(pyrimidin-5-ylmethyl)[(2,2,2- | 9.09(s, 1H), 8.62(s, 2H), 8.09-8.02(m, 2H), | MS 466.1 |
| trifluoroethyl)sulfonyl]amino}- | 7.57(m, 1H), 7.46-7.37(m, 5H), 7.22(m, 1H), | (M⁺ + H). |
| benzyl benzoate | 5.32(s, 2H), 4.92(s, 2H), 3.86(q, 2H) | |
| (3-benzylphenyl)(pyrimidin-5- | 9.11(s, 1H), 8.66(s, 2H), 7.26-7.23(m, 2H), | MS 276.1 |
| ylmethyl)amine | 7.18-7.13(m, 3H), 7.10-7.07(m, 1H), | (M⁺ + H) |
| | 6.60(d, 1H), 6.43(t, 1H), 6.38(d, 1H), | |
| | 4.26(s, 2H), 4.08(br, 1H), 3.87(s, 2H) | |
| N-[3-(2-bromophenoxy)phenyl]- | 9.15(s, 1H), 8.59(s, 2H), 7.34(m, 3H), | MS 505.1 |
| 2,2,2-trifluoro-N-(pyrimidin-5- | 7.17(d, 1H), 6.95(d, 3H), 6.85(s, 1H), | (M⁺ + 2H) |
| ylmethyl)ethanesulfonamide | 4.87(s, 2H), 3.85(q, 2H). | |
| 2,2,2-trifluoro-N-[4-(1- | 9.13(s, 1H), 8.60(s, 2H), 7.10(d, 2H), | MS 418.1 |
| methylbutoxy)phenyl]-N- | 6.84(d, 2H), 4.85(s, 1H), 4.33(m, 1H), | (M⁺ + H) |
| (pyrimidin-5-ylmethyl)ethane- | 3.84(q, 2H), 1.53(m, 1H), 1.39(m, 3H), | |
| sulfonamide | 1.28(d,3H), 0.93(m, 3H). | |
| N-(3-benzylphenyl)-2,2,2-trifluoro- | 9.08(s, 1H), 8.52(s, 2H), 7.29-7.26(m, 3H), | MS 422.1 |
| N-(pyrimidin-5-ylmethyl)ethane- | 7.21-7.16(m, 2H), 7.08-7.05(m, 3H), | (M⁺ + H) |
| sulfonamide | 7.00(s, 1H), 4.83(s, 2H), 3.92(s, 2H), | |
| | 3.78(q, 2H) | |
| N-biphenyl-3-yl-2,2,2-trifluoro-N- | 9.12(s, 1H), 8.63(s, 2H), 7.57(m, 1H), | MS 408.0 |
| (pyrimidin-5-ylmethyl)ethane- | 7.48-7.42(m, 6H), 7.38(m, 1H), 7.18(m, 1H), | (M⁺ + H) |
| sulfonamide | 4.95(s, 2H), 3.87(q, 2H) | |
| 2,2,2-trifluoro-N-(pyrimidin-5- | 9.19(s, 1H), 8.65(s, 2H), 7.21-7.09(m, 3H), | MS 462.1 |
| ylmethyl)-N-(2,2,3,3-tetrafluoro- | 4.91(s, 2H), 3.87(q, 2H). | (M⁺ + H) |
| 2,3-dihydro-1,4-benzodioxin-6- | | |
| yl)ethanesulfonamide | | |
| N-(pyrimidin-5-ylmethyl)aniline | 9.18(s, 1H), 8.78(s, 2H), 7.21(m, 2H), | NA |
| | 6.79(m, 1H), 6.65(d, 2H), 4.41(s, 2H), | |
| | 4.15(br, 1H). | |
| 2,2,2-trifluoro-N-[3-(2- | 9.13(s, 1H), 8.57(s, 2H), 7.27(t, 1H), | MS 454.2 |
| methoxyphenoxy)phenyl]-N- | 7.19(m, 1H0, 7.00(d, 1H), 6.95(m,2H), | (M⁺ + H) |
| (pyrimidin-5-ylmethyl)ethane- | 6.90(d, 2H), 6.79(s, 1H), 4.85(s, 2H), | |
| sulfonamide | 3.82(m, 5H). | |
| 2,2,2-trifluoro-N-(3- | 8.92(s, 1H), 8.54(s, 2H), 7.25(m, 3H), | MS 424.1 |
| phenoxyphenyl)-N-(pyrimidin-5- | 7.08(m, 1H), 7.03(m, 1H), 6.94(t, 1H), | (M⁺ + H) |
| ylmethyl)ethane-sulfonamide | 6.86(m, 1H), 6.81(m, 2H), 4.96(s, 2H), | |
| | 4.38(q, 2H). | |
| [3-(1-methylbutoxy)phenyl]- | 8.90(s, 1H), 8.08-8.10(d, 2H), 7.74-7.76(m, 1H), | NA |
| (quinolin-3-ylmethyl)amine | 7.67-7.68(t, 1H), 7.50-7.51(t, 1H), | |
| | 7.04-7.07(t, 1H), 6.21-6.30(m, 3H), | |
| | 4.49(s, 2H), 4.19-4.31(m, 1H), 4.25(s, 1H), | |
| | 1.66-1.71(m, 1H), 1.41-1.50(m, 3H), | |
| | 1.23-1.24(d, 3H), 0.87-0.90(m, 3H) | |
| N-(quinolin-3-ylmethyl)-3- | ¹H NMR(CDCl₃, 300 MHz) δ 8.83(s, 1H), | MS 319.2 |
| (trifluoromethoxy)aniline | 8.06-8.08(d, 1H), 8.00(s, 1H), 7.70-7.71(d, 1H), | (M⁺ + H) |
| | 7.64-7.67(m, 1H), 7.49-7.51(m, 1H), | |
| | 7.10-7.13(m, 1H), 6.52-6.56(m, 2H), | |
| | 6.48(s, 1H), 4.62(s, 1H), 4.41-4.42(d, 2H) | |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1-22. (canceled)

23. A compound of the formula I: 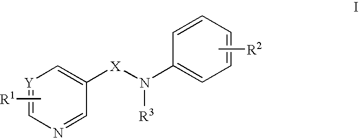
wherein:
X is selected from the group consisting of:
(1) C₁₋₆alkyl, and
(2) a bond;
Y is N to form a pyrimidinyl ring, or
Y is C wheren R¹ at the adjacent position is joined to Y as a phenyl group to form a quinolinyl ring;
R¹ and R² may include multiple substituents and are independently selected from the group consisting of:
(1) hydrogen,
(2) halogen,
(3) C₁₋₆alkyl, unsubstituted or substituted with:
(a) halogen,
(b) —CN,
(c) —COC₁₋₆alkyl
(d) —CO₂C₁₋₆alkyl,
(e) phenyl, which is unsubstituted or substituted with fluoro, bromo, —C₁₋₆alkyl, —CF₃ or —O—C₁₋₆alkyl,
(4) —O—C₁₋₆alkyl, unsubstituted or substituted with halogen,
(5) —O—C₃₋₆cycloalkyl,
(6) phenyl, unsubstituted or substituted with halogen, —C₁₋₆alkyl, —CF₃ or —O—C₁₋₆alkyl,
(7) —O-phenyl, unsubstituted or substituted with halogen, —C₁₋₆alkyl, —CF₃ or —O—C₁₋₆alkyl, and
(8) hydroxyl;
R³ is selected from the group consisting of:
(1) hydrogen,
(2) —SO₂R⁹,
wherein R⁹ is independently selected from:
(a) —C₁₋₆alkyl, which is unsubstituted or substituted with 1-6 halogen or hydroxyl,
(b) —C₃₋₆cycloalkyl, which is unsubstituted or substituted with 1-6 halogen or hydroxyl,
(c) benzyl, and
(d) phenyl,
(3) —CO₂R¹⁰,
wherein R¹⁰ is independently selected from:
(a) hydrogen,
(b) —C₁₋₆alkyl, which is unsubstituted or substituted with 1-6 fluoro,
(c) benzyl, and
(d) phenyl;
or a pharmaceutically acceptable salt thereof.

24. The compound of claim 23 of the formula Ia: 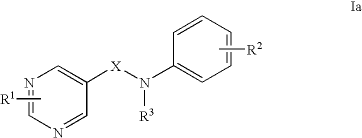
or a pharmaceutically acceptable salt thereof.

25. The compound of claim 23 of the formula Ib: 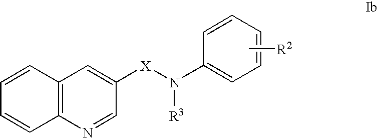
or a pharmaceutically acceptable salt thereof.

26. The compound of claim 23 wherein X is —CH₂—.

27. The compound of claim 23 wherein X is —CH₂CH₂—.

28. The compound of claim 23 wherein R¹ is hydrogen.

29. The compound of claim 23 wherein R² is selected from the group consisting of:
(1) hydrogen,
(2) halogen,
(3) C₁₋₆alkyl, unsubstituted or substituted with halogen or phenyl, which is unsubstituted or substituted with fluoro, bromo, —C₁₋₆alkyl, —CF₃ or —O—C₁₋₆alkyl,
(4) —O—C₁₋₆alkyl, unsubstituted or substituted with halogen,
(5) -phenyl, unsubstituted or substituted with halogen, —C₁₋₆alkyl, —CF₃ or —O—C₁₋₆alkyl, and
(6) —O-phenyl, unsubstituted or substituted with halogen, —C₁₋₆alkyl, —CF₃ or —O—C₁₋₆alkyl.

30. The compound of claim 29 wherein R² is selected from the group consisting of:
(1) hydrogen,
(2) fluoro,
(3) bromo,
(4) C₁₋₆alkyl, unsubstituted or substituted with halogen or phenyl, which is unsubstituted or substituted with fluoro, bromo, —CH₃, —CF₃ or —O—CH₃;
(5) —O—C₁₋₆alkyl, unsubstituted or substituted with halogen,
(6)-phenyl, unsubstituted or substituted with fluoro, bromo, —CH₃, —CF₃, or —O—CH₃, and
(7) —O-phenyl, unsubstituted or substituted with fluoro, bromo, —CH₃, —CF₃, or —O—CH₃.

31. The compound of claim 30 wherein R² is selected from the group consisting of:
(1) hydrogen,
(2) fluoro,
(3) bromo,
(4) -phenyl,
(5) —CH₂-phenyl,
(6) —CH₂-phenyl-CF₃,
(7) —O—CH₃,
(8) —O—CF₃,
(9) —O—CH(CH₃)CH₂CH₂CH₃,
(10) —O-phenyl,
(11) —O-phenyl-F,
(12) —O-phenyl-Br,
(13) —O-phenyl-CH₃, and
(14) —O-phenyl-O—CH₃.

32. The compound of claim 23 wherein R³ is hydrogen.

33. The compound of claim 23 wherein R³ is —SO₂—C₁₋₆alkyl, which is unsubstituted or substituted with fluoro.

34. The compound of claim 33 wherein R³ is —SO₂—CH₂CF₃.

35. A compound which is selected from the group consisting of:
N-[3-(cyclopentyloxy)phenyl]-2,2,2-trifluoro-N-(pyrimidin-5-ylmethyl)ethane-sulfonamide;
2,2,2-trifluoro-N-(pyrimidin-5-ylmethyl)-N-{3-[2-(trifluoromethyl)benzyl]phenyl}ethanesulfonamide;
2,2,2-trifluoro-N-[3-(2-methoxyphenoxy)phenyl]-N-(pyrimidin-5-ylmethyl)ethane-sulfonamide;
3-{(pyrimidin-5-ylmethyl)[(2,2,2-trifluoroethyl)sulfonyl]amino}benzyl benzoate;
[3-(1-methylbutoxy)phenyl](quinolin-3-ylmethyl)amine;
2,2,2-trifluoro-N-[3-(1-methylbutoxy)phenyl]-N-(pyrimidin-5-ylmethyl)ethane-sulfonamide;
2,2,2-trifluoro-N-(pyrimidin-5-ylmethyl)-N-{3-[2-(trifluoromethyl)benzyl]phenyl}-ethanesulfonamide;
2,2,2-trifluoro-N-(pyrimidin-5-ylmethyl)-N-[3-(trifluoromethoxy)-phenyl]ethane-sulfonamide;
N-(3-ethoxyphenyl)-2,2,2-trifluoro-N-(pyrimidin-5-ylmethyl)ethane-sulfonamide;
2,2,2-trifluoro-N-(3-methoxy-phenyl)-N-(pyrimidin-5-ylmethyl)ethanesulfonamide;
N-[3-({[tert-butyl(dimethyl)-silyl]oxy}methyl)phenyl]-2,2,2-trifluoro-N-(pyrimidin-5-ylmethyl)ethanesulfonamide;
2,2,2-trifluoro-N-[3-(phenoxymethyl)phenyl]-N-(pyrimidin-5-ylmethyl)ethane-sulfonamide;
N-[3-(cyclopentyloxy)phenyl]-2,2,2-trifluoro-N-(pyrimidin-5-ylmethyl)ethane-sulfonamide;
2,2,2-trifluoro-N-[3-(hydroxymethyl)phenyl]-N-(pyrimidin-5-ylmethyl)ethane-sulfonamide;
2,2,2-trifluoro-N-[3-(2-methylphenoxy)phenyl]-N-(pyrimidin-5-ylmethyl)ethane-sulfonamide;
2,2,2-trifluoro-N-[3-(2-fluorophenoxy)phenyl]-N-(pyrimidin-5-ylmethyl)ethane-sulfonamide;
2,2,2-trifluoro-N-(4-phenoxyphenyl)-N-(pyrimidin-5-ylmethyl)ethanesulfonamide;
2,2,2-trifluoro-N-(pyrimidin-5-ylmethyl)-N-{3-[(trifluoromethyl)-thio]phenyl}-ethanesulfonamide;
3-{(pyrimidin-5-ylmethyl)[(2,2,2-trifluoroethyl)sulfonyl]amino}benzyl benzoate;
N-[3-(2-bromophenoxy)phenyl]-2,2,2-trifluoro-N-(pyrimidin-5-ylmethyl)ethane-sulfonamide;
2,2,2-trifluoro-N-[4-(1-methylbutoxy)phenyl]-N-(pyrimidin-5-ylmethyl)ethane-sulfonamide;
N-(3-benzylphenyl)-2,2,2-trifluoro-N-(pyrimidin-5-ylmethyl)ethane-sulfonamide;
N-biphenyl-3-yl-2,2,2-trifluoro-N-(pyrimidin-5-ylmethyl)ethane-sulfonamide;
2,2,2-trifluoro-N-(pyrimidin-5-ylmethyl)-N-(2,2,3,3-tetrafluoro-2,3-dihydro-1,4-benzodioxin-6-yl)ethanesulfonamide;
2,2,2-trifluoro-N-[3-(2-methoxyphenoxy)phenyl]-N-(pyrimidin-5-ylmethyl)ethane-sulfonamide;
2,2,2-trifluoro-N-(3-phenoxyphenyl)-N-(pyrimidin-5-ylmethyl)ethanesulfonamide;
[3-(1-methylbutoxy)phenyl]-(quinolin-3-ylmethyl)amine;
N-(quinolin-3-ylmethyl)-3-(trifluoromethoxy)aniline;
or a pharmaceutically acceptable salt thereof.

36. A pharmaceutical composition which comprises an inert carrier and a compound of claim 23 or a pharmaceutically acceptable salt thereof.

37. A method for treating, controlling, ameliorating or reducing the risk of a neurological and psychiatric disorders associated with glutamate dysfunction in a mammalian patient in need of such which comprises administering to the patient a therapeutically effective amount of a compound of claim 23 or a pharmaceutically acceptable salt thereof.

38. A method for treating, controlling, ameliorating or reducing the risk of anxiety in a mammalian patient in need of such which comprises administering to the patient a therapeutically effective amount of a compound of claim 23 or a pharmaceutically acceptable salt thereof.

39. A method for treating, controlling, ameliorating or reducing the risk of depression in a mammalian patient in need of such which comprises administering to the patient a therapeutically effective amount of a compound of claim 23 or a pharmaceutically acceptable salt thereof.

40. A method for treating, controlling, ameliorating or reducing the risk of migraine in a mammalian patient in need of such which comprises administering to the patient a therapeutically effective amount of a compound of claim 23 or a pharmaceutically acceptable salt thereof.

41. A method for treating, controlling, ameliorating or reducing the risk of schizophrenia in a mammalian patient in need of such which comprises administering to the patient a therapeutically effective amount of a compound of claim 23 or a pharmaceutically acceptable salt thereof.

42. A method for treating, controlling, ameliorating or reducing the risk of epilepsy in a mammalian patient in need of such which comprises administering to the patient a therapeutically effective amount of a compound of claim 23 or a pharmaceutically acceptable salt thereof.
